(19) United States
(12) Patent Application Publication
(10) Pub. No.: US 2023/0025575 A1
(43) Pub. Date: Jan. 26, 2023

(54) PROCESS FOR PREPARING AMINES OVER A COPPER CATALYST

(71) Applicant: BASF SE, Ludwigshafen am Rhein (CN)

(72) Inventors: Martin ERNST, Ludwigshafen am Rhein (DE); Ansgar Gereon ALTENHOF, Ludwigshafen am Rhein (DE); Hermann LUYKEN, Ludwigshafen am Rhein (DE); Tatjana HUBER, Ludwigshafen am Rhein (DE); Sebastian HAUPT, Ludwigshafen am Rhein (DE); Dieter KOLASSA, Ludwigshafen am Rhein (DE)

(21) Appl. No.: 17/781,928

(22) PCT Filed: Nov. 24, 2020
(86) PCT No.: PCT/EP2020/083162

§ 371 Date: Jun. 2, 2022

(30) Foreign Application Priority Data
Dec. 3, 2019 (EP) 19213226.4

(51) Int. Cl. C07C 209/60; B01J 23/72; B01J 23/10; B01J 23/89; B01J 21/04

(52) U.S. Cl. CPC C07C 209/60; B01J 23/72; B01J 23/10; B01J 23/8993; B01J 21/04

(57) ABSTRACT

A process for preparing an amine by reacting an aldehyde and/or ketone with a nitrogen compound selected from the group consisting of ammonia and primary and secondary amines, and subsequent hydrogenation of the resulting reaction product in the liquid phase and in the presence of hydrogen and a heterogeneous copper oxide hydrogenation catalyst at a temperature of 20 to 230° C., wherein the aldehyde and/or ketone is reacted with the nitrogen compound either together with the hydrogenation in the liquid phase and in the presence of the hydrogen and of the catalyst (alternative 1) or in a step preceding the hydrogenation (alternative 2), and wherein the catalytically active composition of the catalyst, prior to reduction thereof with hydrogen, comprises at least 24% by weight of oxygen compounds of copper, calculated as Cu.

### BACKGROUND OF THE INVENTION

The present invention relates to a process for preparing an amine by reacting an aldehyde and/or ketone with a nitrogen compound selected from the group consisting of ammonia and primary and secondary amines, and subsequently hydrogenating the resulting reaction product in the liquid phase and in the presence of hydrogen and of a heterogeneous copper oxide hydrogenation catalyst.

### PRIOR ART

The uses of the process products include use as intermediates in the production of fuel additives (U.S. Pat. No. 3,275,554; DE-A-21 25 039 and DE-A-36 11 230) and biologically active substances (Mokrov G. V. et al, Russian Chemical Bulletin, 59(6), 1254-1266, 210), or as crosslinkers in polyurethane foams (U.S. Pat. No. 8,552,078 B2).

WO 2004/085353 A1 (BASF Aktiengesellschaft) describes the preparation of hydrogenation catalysts comprising, inter alia, CuO, Al₂O₃, La₂O₃ and elemental copper. Such catalysts are used for hydrogenation of organic compounds having at least one carbonyl group.

WO 2007/006719 A1 (BASF Aktiengesellschaft) describes the preparation of hydrogenation catalysts comprising, inter alia, CuO, Al₂O₃, La₂O₃ and elemental copper. The stability of the catalyst is increased by treatment with boiling water and/or steam. Such catalysts are used for hydrogenation of organic compounds having at least one carbonyl group.

WO 2007/107477 A1 (BASF Aktiengesellschaft) describes the preparation of an amine by reaction of an aldehyde and/or ketone with hydrogen and a nitrogen compound in the presence of an eggshell catalyst that preferably comprises Pd/Ag/Al₂O₃.

WO 2010/031719 A1 (BASF SE) describes the preparation of an amine by reacting an aldehyde and/or ketone with hydrogen and a nitrogen compound over a catalyst containing copper and aluminum oxide. The copper oxide content, calculated as CuO, may be well above 50% by weight. The reaction takes place exclusively in the gas phase.

WO 2011/067199 A1 (BASF SE) describes the preparation of a by reaction of an aldehyde and/or ketone with hydrogen and a nitrogen compound over a supported copper, nickel, cobalt and tin catalyst, wherein the support is aluminum oxide (Al₂O₃). What is disclosed is a catalyst having a content of copper oxide, calculated as CuO, of not more than 20% by weight.

U.S. Pat. No. 8,552,078 B2 (Air Products and Chemicals, Inc.) describes the reaction of polyamines with suitable aldehydes and ketones, for example the reaction of 1,2-EDA with benzaldehyde to form N-benzylethylene-1,2-diamine. The catalyst used here is Pd/C.

WO 2016/023839 A1 (Sika Technology AG) describes the reaction of 1,2-PDA with an appropriate aldehyde or ketone (for example the reaction with benzaldehyde to form N′-benzylpropylene-1,2-diamine). The catalyst used here is Pd/C.

WO 2017/037069 A1 (Sika Technology AG) describes the reaction of 1,2-EDA with an appropriate aldehyde or ketone (for example the reaction with benzaldehyde to form N-benzylethane-1,2-diamine). A further product that occurs is mainly polyalkylated 1,2-EDA (for example N,N′-benzylethylene-1,2-diamine). The catalyst used here is Pd/C.

In the case of preparation of diamines, for example N-benzylethylene-1,2-diamine (NBEDA) or N-benzylpropylene-1,2-diamine (NBPDA), the prior art describes exclusively the use of Pd/C as useful catalysts. The specifically disclosed processes are based here on preparation on the laboratory scale. However, the catalysts used are not directly suitable for use in industrial scale processes. A disadvantage here is that the catalyst has to be used in correspondingly large amounts. But Pd is a material that occurs only rarely on earth and is thus of limited availability. The high procurement costs for such catalysts consequently reduce the economic viability of a corresponding production process. Further problems arise with regard to the service life and mechanical stability of the catalysts, which is insufficient for an industrial scale process. For instance, an activated carbon support does not have sufficient stability and hence service life.

### OBJECT

It was an object of the present invention to improve the economic viability of existing processes for reductive amination of aldehydes and ketones and to remedy one or more disadvantages of the prior art, especially the abovementioned disadvantages. The intention was to find catalysts which are preparable industrially in a simple manner, and which allow the abovementioned aminations to be conducted with high conversion, high yield, space-time yield (STY), selectivity coupled with high mechanical stability of the shaped catalyst body, and low “runaway risk” (the triggering of thermal runaway reactions). The catalysts were accordingly to have high activity and, under the reaction conditions, high chemical and mechanical stability and long service life.

[Space-time yields are expressed in ‘Amount of product/(Catalyst volume·Time)’(kg/(I_{cat}·h)) and/or ‘Amount of product/(Reactor volume·Time)’(kg/I_{reactor}·h))].

### DESCRIPTION OF THE INVENTION

Surprisingly, a process has been found for preparing an amine by reacting an aldehyde and/or ketone with a nitrogen compound selected from the group consisting of ammonia and primary and secondary amines, and subsequent hydrogenation of the resulting reaction product in the liquid phase and in the presence of hydrogen and a heterogeneous copper oxide hydrogenation catalyst at a temperature of 20 to 230° C., wherein the aldehyde and/or ketone is reacted with the nitrogen compound either together with the hydrogenation in the liquid phase and in the presence of the hydrogen and of the catalyst (alternative 1) or in a step preceding the hydrogenation (alternative 2), and wherein the catalytically active composition of the catalyst, prior to reduction thereof with hydrogen, comprises at least 24% by weight of oxygen compounds of copper, calculated as Cu.

It has been found that, with the aid of such a catalyst, corresponding amines can be prepared in high yield and selectivity. This is surprising to the person skilled in the art at least on account of the consideration that follows. According to WO 2004/085353 A1 and WO 2007/006719 A1 (both BASF Aktiengesellschaft), a corresponding catalyst is used exclusively for hydrogenation of organic compounds having carbonyl groups. The person skilled in the art would thus have actually expected the aldehyde/ketone to be reduced to the corresponding alcohol under the given reaction conditions and hence not to react, or to do so only to a small degree, with the nitrogen compound to give the desired product amine.

It was likewise surprising that such catalysts are suitable at all for amination in the liquid phase, or give better results in the liquid phase than in the gas phase. This is surprising especially in the light of WO 2010/031719 A1. It is taught therein at page 10 lines 22 to 26 that the procedure according to WO 2010/031719 A1 (amination in the gas phase over a catalyst comprising oxygen compounds of copper and aluminum) gives, inter alia, better selectivities than a synthesis in the liquid phase.

### Catalyst

According to the invention, a heterogeneous copper oxide hydrogenation catalyst is used, the catalytically active composition of which, prior to reduction thereof with hydrogen, comprises at least 24% by weight, preferably at least 40% by weight, of oxygen compounds of copper, calculated as Cu.

The catalytically active composition of the catalyst, after the final heat treatment thereof and prior to the reduction thereof with hydrogen, is defined as the sum total of the masses of the catalytically active constituents. The catalytically active constituents are either metals in elemental form or the oxygen compounds thereof.

The concentration figures (in % by weight) of the catalytically active constituents of the catalyst are each based on the catalytically active composition of the finished catalyst after the last heat treatment thereof (calcination) and before the reduction thereof with hydrogen. They further relate to the mass of the corresponding metal, irrespective of whether the metal is in elemental form or in the form of an oxygen compound, where the mass of the corresponding metal is based on the total mass of all metals present in the catalytically active composition. If the catalytically active constituent in question is not a metal (in elemental form) but the oxygen compound of a metal, this is illustrated by the addition “calculated as . . . ”. For example: “oxygen compounds of copper, calculated as Cu”, etc.

The catalytically active composition of the catalyst, prior to reduction thereof with hydrogen, comprises preferably in the range from 24% to 98% by weight, more preferably 50% to 90% by weight, most preferably 55% to 85% by weight or even 60% to 80% by weight, of oxygen compounds of copper, calculated as Cu.

The catalytically active composition of the catalyst, prior to reduction thereof with hydrogen, preferably comprises in the range from 0.5% to 75% by weight, more preferably 0.5% to 40% by weight, most preferably 1% to 35% by weight or even 1.5% to 30% by weight or 1.5% to 20% by weight, of oxygen compounds of aluminum, calculated as Al.

For example, the catalytically active composition of the catalyst, prior to reduction thereof with hydrogen, may also comprise 24% to 98% by weight, preferably 40% to 95% by weight or even 50% to 90% by weight, of oxygen compounds of copper, calculated as Cu, and 0.5% to 75% by weight, preferably 4% to 59% by weight or even 9% to 49% by weight, of oxygen compounds of aluminum, calculated as Al.

According to the invention, it is possible to use a catalyst, the main constituent of which is oxygen compounds of Cu and of Al. In this case, the sum total of these two constituents, calculated as Cu and Al, of the catalytically active composition of the catalyst is typically 70% to 100% by weight, preferably 75% to 100% by weight, more preferably 80% to 100% by weight. Further components may, as set out further down, be oxygen compounds of lanthanum, tungsten, molybdenum, titanium and zirconium, and elemental copper.

With regard to mechanical stability, it is advantageous when the catalyst of the invention comprises the constituents specified on the pages that follow (especially oxygen compounds of lanthanum, tungsten, molybdenum, titanium and zirconium, elemental copper, and oxygen compounds of magnesium, calcium, silicon and iron).

The catalytically active composition of the catalyst, prior to reduction thereof with hydrogen, preferably comprises in the range from 0.5% to 40% by weight, more preferably 1% to 35% by weight and most preferably 1.5% to 30% by weight or even 1.5% to 20% by weight of at least one oxygen compound selected from the group consisting of oxygen compounds of lanthanum, tungsten, molybdenum, titanium and zirconium, calculated as La, W, M, Ti, and Zr, preference being given to oxygen compounds of lanthanum.

In particular, the catalytically active composition of the catalyst, prior to reduction thereof with hydrogen, preferably comprises in the range from 0.5% to 40% by weight, more preferably 1% to 35% by weight and most preferably 1.5% to 30% by weight or even 1.5% to 20% by weight of oxygen compounds of lanthanum, calculated as La, where the total concentration of oxygen compounds of lanthanum and of any oxygen compounds of tungsten, molybdenum, titanium and zirconium present, respectively calculated as W, M, Ti and Zr, is within the aforementioned ranges. If, for example, the upper limit of 40% by weight of oxygen compounds of lanthanum is attained, this means that the catalyst does not comprise any oxygen compounds of tungsten, molybdenum, titanium and/or zirconium.

The catalytically active composition of the catalyst, prior to reduction thereof with hydrogen, comprises preferably in the range from 0.1% to 40% by weight, more preferably 1% to 35% by weight and most preferably 1.5% to 30% by weight or even 1.5% to 20% by weight of elemental copper and/or in the range from 0.1% to 40% by weight, 0.5% to 35% by weight and more preferably 1% to 30% by weight or even 1.5% to 20% by weight of at least one oxygen compound selected from the group consisting of oxygen compounds of magnesium, calcium, silicon and iron, calculated as Mg, Ca, Si and Fe, particular preference being given to elemental copper. Most preferably, the catalyst comprises elemental copper, but none of the oxygen compounds of magnesium, calcium, silicon and iron mentioned here.

Elemental copper may become part of the catalyst in step (ii) of the preparation process described below. The same is true of the abovementioned oxygen compounds of magnesium, calcium, silicon and iron when cement is used in step (ii).

More particularly, the catalytically active composition of the catalyst, prior to reduction thereof with hydrogen, may include, in a proportion of not more than 10% by weight, preferably not more than 8% by weight, more preferably not more than 5% by weight or even not more than 4% or not more than 3% by weight, at least one further component selected from the group consisting of the elements Re, Fe, Ru, Co, Rh, Ir, No, Pd and Pt.

Such further components may be part of the oxidic material described below and may thus become part of the catalyst in step (i) of the preparation process described below.

The catalytically active composition of the catalysts of the invention and of those used in the process of the invention preferably does not comprise any silver, in each case either in metallic form (oxidation state=0) or in an ionic form (oxidation state≠0), especially oxidized form.

The catalytically active composition of the catalyst preferably does not comprise any oxygen compounds of chromium.

In a particularly preferred embodiment, the catalytically active composition of the catalysts used in the process of the invention does not comprise any further catalytically active components other than those mentioned explicitly above, either in elemental form (oxidation state=0) or in ionic form (oxidation state≠0).

Typically, the catalytically active composition has not been doped with further metals or metal compounds. However, this preferably excludes customary accompanying trace elements that originate from the metal beneficiation of copper, aluminum, lanthanum, tungsten, molybdenum, titanium and zirconium, and any magnesium, calcium, silicon and iron.

In the process of the invention, the catalysts are used preferably in the form of catalysts consisting solely of catalytically active composition and optionally a shaping auxiliary that does not form part of the catalytically active composition (for example graphite or stearic acid) if the catalyst is used in the form of shaped bodies, i.e. not comprising any other catalytically active substances.

In a preferred embodiment, the catalytically active composition of the catalyst, prior to reduction thereof with hydrogen, comprises in the range from
50% to 90% by weight, more preferably 55% to 85% by weight and most preferably 60% to 80% by weight of oxygen compounds of copper, calculated as Cu,
0.5% to 40% by weight, more preferably 1% to 35% by weight and most preferably 1.5% to 30% by weight or even 1.5% to 20% by weight of oxygen compounds of aluminum, calculated as Al,
0.5% to 40% by weight, more preferably 1% to 35% by weight and most preferably 1.5% to 30% by weight or even 1.5% to 20% by weight of at least one oxygen compound selected from the group consisting of oxygen compounds of lanthanum, tungsten, molybdenum, titanium and zirconium, calculated as La, W, Mo, Ti and Zr, and
0.1% to 40% by weight, more preferably 0.5% to 35% by weight and most preferably 1% to 30% by weight or even 1% to 20% by weight of elemental copper.

In a particularly preferred embodiment, the catalytically active composition of the catalyst, prior to reduction thereof with hydrogen, comprises in the range from
50% to 90% by weight, more preferably 55% to 85% by weight and most preferably 60% to 80% by weight of oxygen compounds of copper, calculated as Cu,
0.5% to 40% by weight, more preferably 1% to 35% by weight and most preferably 1.5% to 30% by weight or even 1.5% to 20% by weight of oxygen compounds of aluminum, calculated as Al,
0.5% to 40% by weight, more preferably 1% to 35% by weight and most preferably 1.5% to 30% by weight or even 1.5% to 20% by weight of oxygen compounds of lanthanum, calculated as La, and
0.1% to 40% by weight, more preferably 0.5% to 35% by weight and most preferably 1% to 30% by weight or even 1% to 20% by weight of elemental copper.

Typically, in this particularly preferred embodiment, the total concentration of oxygen compounds of lanthanum and of any oxygen compounds of tungsten, molybdenum, titanium and zirconium present, respectively calculated as W, M, Ti and Zr, is within the aforementioned ranges.

The sum total of the constituents of the catalytically active composition that are mentioned above in the preferred and particularly preferred embodiments is typically 70% to 100% by weight, preferably 80% to 100% by weight, more preferably 90% to 100% by weight, particularly >95% by weight, very particularly >98% by weight, especially >99% by weight, for example especially preferably 100% by weight.

The catalyst of the invention is preferably preparable by a process in which
(i) an oxidic material comprising oxygen compounds of copper and of aluminum and at least one oxygen compound selected from the group consisting of oxygen compounds of lanthanum, tungsten, molybdenum, titanium and zirconium, preference being given to oxygen compounds of lanthanum, is provided,
(ii) pulverulent metallic copper, copper flakes, pulverulent cement or a mixture thereof, preferably pulverulent metallic copper, copper flakes or a mixture thereof, is added to the oxidic material,
(iii) the mixture resulting from (ii) is shaped to give the copper oxide catalyst and is preferably then calcined at least once.

The amount of the materials used in steps (i) and (ii) should be chosen such that the catalyst of the invention is of the composition as described further up.

Preference is accordingly also given to a process in which, in a process step preceding the amination, the catalyst is first prepared by the process described above. A catalyst thus prepared is notable for particularly high mechanical stability.

The cement used is preferably an alumina cement. The alumina cement more preferably consists essentially of aluminum oxide and calcium oxide; it more preferably consists of 75% to 85% by weight of aluminum oxide and 15% to 25% by weight of calcium oxide. In addition, it is possible to use a cement based on magnesium oxide/aluminum oxide, calcium oxide/silicon oxide and calcium oxide/aluminum oxide/iron oxide.

The oxidic material may include at least one further component selected from the group consisting of the elements Re, Fe, Ru, Co, Rh, Ir, No, Pd and Pt. The respective amount of these components in the oxidic material should be chosen such that the appropriate amount is present in the catalytically active composition of the catalyst within the above-designated ranges.

In an embodiment that is especially preferred, the catalytically active composition of the catalyst, prior to reduction thereof with hydrogen, comprises in the range from
50% to 90% by weight, more preferably 55% to 85% by weight and most preferably 60% to 80% by weight of oxygen compounds of copper, calculated as Cu,
0.5% to 40% by weight, more preferably 1% to 35% by weight and most preferably 1.5% to 30% by weight or even 1.5% to 20% by weight of oxygen compounds of aluminum, calculated as Al,
0.5% to 40% by weight, more preferably 1% to 35% by weight and most preferably 1.5% to 30% by weight or even 1.5% to 20% by weight of at least one oxygen compound selected from the group consisting of oxygen compounds of lanthanum, tungsten, molybdenum, titanium and zirconium, calculated as La, W, Mo, Ti and Zr, and
0.1% to 40% by weight, more preferably 0.5% to 35% by weight and most preferably 1% to 30% by weight or even 1% to 20% by weight of elemental copper, and is preparable by a process in which
(i) an oxidic material comprising oxygen compounds of copper and of aluminum and at least one oxygen compound selected from the group consisting of oxygen compounds of lanthanum, tungsten, molybdenum, titanium and zirconium is provided,
(ii) pulverulent metallic copper, copper flakes or a mixture thereof is added to the oxidic material,
(iii) the mixture resulting from (ii) is shaped to give the copper oxide catalyst and is preferably then calcined at least once.

In an embodiment that is very especially preferred, the catalytically active composition of the catalyst, prior to reduction thereof with hydrogen, comprises in the range from
50% to 90% by weight, more preferably 55% to 85% by weight and most preferably 60% to 80% by weight of oxygen compounds of copper, calculated as Cu,
0.5% to 40% by weight, more preferably 1% to 35% by weight and most preferably 1.5% to 30% by weight or even 1.5% to 20% by weight of oxygen compounds of aluminum, calculated as Al,
0.5% to 40% by weight, more preferably 1% to 35% by weight and most preferably 1.5% to 30% by weight or even 1.5% to 20% by weight of oxygen compounds of lanthanum, calculated as La, and
0.1% to 40% by weight, more preferably 0.5% to 35% by weight and most preferably 1% to 30% by weight or even 1% to 20% by weight of elemental copper, and is preparable by a process in which
(i) an oxidic material comprising oxygen compounds of copper and aluminum and oxygen compounds of lanthanum is provided,
(ii) pulverulent metallic copper, copper flakes or a mixture thereof is added to the oxidic material,
(iii) the mixture resulting from (ii) is shaped to give the copper oxide catalyst and is preferably then calcined at least once.

Typically, in this embodiment that is very especially preferred, the total concentration of oxygen compounds of lanthanum and of any oxygen compounds of tungsten, molybdenum, titanium and zirconium present, respectively calculated as W, M, Ti and Zr, is within the aforementioned ranges.

The sum total of the constituents of the catalytically active composition that are mentioned above in the embodiment that is especially and very especially preferred is typically 70% to 100% by weight, preferably 80% to 100% by weight, more preferably 90% to 100% by weight, particularly >95% by weight, very particularly >98% by weight, especially >99% by weight, for example especially preferably 100% by weight.

In preferred embodiments, the catalysts of the invention are used in the form of all-active catalysts, impregnated catalysts, eggshell catalysts and precipitated catalysts. The catalyst of the invention is preferably not supported.

The catalyst used in the process of the invention may especially have the feature that the copper component, the aluminum component and the component of at least one oxygen compound of lanthanum, tungsten, molybdenum, titanium or zirconium are preferably precipitated simultaneously or successively with a soda solution, then dried, calcined, tableted and calcined once again.

A particularly useful precipitation method is as follows:
A) A copper salt solution, an aluminum salt solution and a solution of at least one salt of lanthanum, tungsten, molybdenum, titanium or zirconium, or a solution comprising copper salts, aluminum salts and at least one of the salts of lanthanum, tungsten, molybdenum, titanium or zirconium, is precipitated in parallel or successively with a soda solution. The precipitated material subsequently dried and optionally calcined.
A) Precipitation of a copper salt solution and a solution of at least one salt of lanthanum, tungsten, molybdenum, titanium or zirconium, or a solution comprising copper salt and at least one salt of lanthanum, tungsten, molybdenum, titanium or zirconium, onto a prefabricated aluminum oxide support. In a particularly preferred embodiment, this takes the form of a powder in an aqueous suspension. The support material may alternatively take the form of spheres, extrudates, spall or tablets.
B1) In one embodiment (I), a copper salt solution and a solution of at least one salt of lanthanum, tungsten, molybdenum, titanium or zirconium, or a solution comprising copper salt and at least one salt of lanthanum, tungsten, molybdenum, titanium or zirconium, is precipitated, preferably with soda solution. The initial charge used is an aqueous suspension of the aluminum oxide support material.

Precipitated solids that result from A) or B) are filtered in a customary manner and preferably washed to free them of alkali, as described, for example, in DE 198 09 418.3.

Both the end products from A) and those from B) are dried at temperatures of 50 to 150° C., preferably at 120° C., and optionally calcined thereafter, preferably at generally 200 to 600° C., especially at 300 to 500° C., for 2 hours.

Starting substances used for A) and/or B) may in principle be any of the Cu(I) and/or Cu(II) salts that are soluble in the solvents used in the application, for example nitrates, carbonates, acetates, oxalates or ammonium complexes, analogous aluminum salts, and salts of lanthanum, tungsten, molybdenum, titanium or zirconium. Particular preference is given to using copper nitrate for processes according to A) and B).

In the process of the invention, the above-described dried and optionally calcined powder is preferably processed to tablets, rings, ring tablets, extrudates, honeycombs or similar shaped bodies. For this purpose, all the suitable methods known from the prior art are conceivable.

It is a feature of the catalysts thus prepared that the addition of lanthanum, tungsten, molybdenum, titanium or zirconium in the precipitation leads to a high stability of the catalyst.

A further increase in the stability of the catalyst is achieved by the addition of pulverulent metallic copper or copper flakes and cement in step (ii).

Preferably, graphite is added to the oxidic material and/or the mixture resulting from (ii) in a total amount of 0.5% to 5% by weight, based on the total weight of the oxidic material. This shall be understood to mean that the total amount added is within the stated range, meaning that, for example, 1% by weight is added to the oxidic material and a further 2% by weight of graphite to the mixture resulting from (ii) (cf. also example 1).

After addition of the copper powder, the copper flakes or optionally the cement powder or the mixture thereof and optionally graphite to the oxidic material, the catalyst obtained after the shaping is typically calcined at least once over a period of generally 0.5 to 10 h, preferably 0.5 to 2 hours. The temperature in this at least one calcination step is generally in the range from 200 to 600° C., preferably in the range from 250 to 500° C. and more preferably in the range from 270 to 400° C.

In the case of shaping with cement powder, it may be advantageous to moisten the shaped body obtained prior to the calcination with water and then to dry it.

In order to further improve the stability of the catalyst, in an additional step (iv), the copper oxide catalyst obtained in step (iii), as described in WO 2007/006719 A1 (BASF Aktiengesellschaft), may be treated with boiling water and/or steam.

According to the invention, it is likewise possible to use a catalyst consisting essentially of oxygen compounds of Cu and of Al. In this case, the sum total of these two constituents, calculated as Cu and Al, of the catalytically active composition of the catalyst is typically 90% to 100% by weight, preferably 98% to 100% by weight, more preferably 99% by weight, most preferably 100% by weight.

The preparation of such catalysts consisting essentially of oxygen compounds of Cu and of Al is possible by various methods. The catalysts are obtainable, for example, by peptizing pulverulent mixtures of the hydroxides, carbonates, oxides and/or other salts of the aluminum and copper components with water, and subsequently extruding and heat-treating the resultant material.

The catalysts used in the process of the invention may also be prepared by impregnation of aluminum oxide (Al₂O₃), for example in the form of powder or shaped tablets. Aluminum oxide may be used in various polymorphs; preference is given to α-(alpha), γ-(gamma) or Θ-Al₂O₃ (theta-Al₂O₃). Particular preference is given to using γ-Al₂O₃.

Shaped bodies of aluminum oxide can be produced by the customary methods. The catalyst preferably has a tablet shape having a diameter in the range from 1 to 4 mm and a height in the range from 1 to 4 mm.

In principle, the preparation of such catalysts consisting essentially of oxygen compounds of Cu and of Al is known to the person skilled in the art and is described, for example, in WO 2010/031719 A1 (BASF SE).

For activation, the catalyst of the invention is subjected to preliminary reduction with hydrogen, preferably hydrogen-inert gas mixtures, especially hydrogen/nitrogen mixtures, at temperatures in the range from 100 to 500° C., preferably in the range from 150 to 350° C. and especially in the range from 180 to 200° C. Preference is given here to a mixture having a hydrogen content in the range from 1% to 100% by volume, more preferably in the range from 1% to 50% by volume.

In a preferred embodiment, the catalyst of the invention, prior to use thereof, is activated in a manner known per se by treatment with hydrogen. The activation is either effected beforehand in a reduction oven or after installation in the reactor. If the reactor has been activated beforehand in the reduction oven, it is installed into the reactor and charged directly under hydrogen pressure with the further reactants: nitrogen compound and aldehyde and/or ketone. If it has been reduced and surface passivated in the reduction oven, it can be charged with the reactants either without further reductive treatment with hydrogen or after further treatment with hydrogen in the reactor.

### Process Regime

Unless stated otherwise, all pressure figures refer to absolute pressure.

The process of the invention can be conducted continuously or batchwise, preference being given to a continuous mode of operation.

The process of the invention can be operated in one stage (alternative 1) or two stages (alternative 2). The resulting reaction product is typically an imine or enamine. This is hydrogenated in the presence of hydrogen and the catalyst.

In alternative 1, the aldehyde and/or ketone is reacted with the nitrogen compound together with the hydrogenation in the liquid phase and in the presence of the hydrogen and the catalyst. Accordingly, reaction of aldehyde and/or ketone with the nitrogen compound and the hydrogenation take place under the same reaction conditions. In other words, all the statements made with regard to the hydrogenation conditions are likewise applicable to the reaction of the aldehyde and/or ketone with the nitrogen compound.

In alternative 2, the aldehyde and/or ketone is reacted with the nitrogen compound in a step preceding the hydrogenation. In this case, the aldehyde and/or ketone is reacted with the nitrogen compound in the absence of hydrogen and catalyst to give the resulting reaction product. This is hydrogenated in a subsequent step in the presence of hydrogen and the catalyst.

In alternative 2, the aldehyde or ketone is reacted with the nitrogen component generally at pressures of 0.1 to 30 MPa, preferably 0.1 to 25 MPa, more preferably 0.1 to 21 MPa, and temperatures of generally 10 to 250° C., particularly 15 to 240° C., preferably 20 to 230° C., more preferably 25 to 220° C., especially 30 to 210° C. For the hydrogenation of the resulting reaction product, preference is given to the temperatures and pressures mentioned below in connection with the operations of the invention in the liquid phase.

In alternative 1, the amine is prepared by reacting the aldehyde and/or ketone and the nitrogen compound together with the hydrogenation in the liquid phase and in the presence of the hydrogen and the catalyst. This hydrogenation of the resulting reaction product from the reaction of the aldehyde or ketone with the nitrogen compound is effected in situ. A procedure according to alternative 1 is preferred. In this case, the reaction and subsequent hydrogenation are effected under the same conditions.

When working in the liquid phase in accordance with the invention, the reactants (aldehyde or ketone plus nitrogen component) (alternative 1) or the reaction product of the invention from the reaction of aldehyde and/or ketone with the nitrogen component (alternative 2) are contacted with the catalyst simultaneously in the liquid phase at pressures of generally 1 to 30 MPa (10-300 bar), preferably 2 to 25 MPa, more preferably 3 to 20 MPa, and temperatures of 20 to 230° C., particularly 30 to 220° C., preferably 40 to 210° C., more preferably 50 to 200° C., especially 60 to 190° C., including hydrogen. The catalyst is present typically in an adiabatic or externally cooled reactor, especially a fixed bed reactor, for example a shell and tube reactor in the case of a continuous reaction regime or an autoclave in the case of a batchwise reaction regime. In the case of a continuous reaction regime, either trickle mode or liquid-phase mode is possible. In a continuous reaction regime, the catalyst hourly space velocity is generally in the range from 0.05 to 5, preferably 0.1 to 2, more preferably 0.2 to 0.6, kg of aldehyde or ketone (alternative 1) or reaction product (alternative 2) per liter of catalyst (bed volume) and hour. Both in the continuous and in the batchwise reaction regime, it is optionally possible to dilute the reaction product or the reactants with a suitable solvent, such as tetrahydrofuran, dioxane, N-methylpyrrolidone, methanol, isopropanol or ethylene glycol dimethyl ether. In the case of the continuous reaction regime, it is appropriate to heat the reactants even before they are fed into the reactor, preferably to the reaction temperature. In the continuous reaction regime, the reaction is preferably effected without solvent.

In a continuous mode of operation, the hydrogenation can be effected in a reactor, typically a fixed bed reactor, for example in an isothermal or adiabatic manner, where, in the case of an isothermal reaction regime for both alternatives, the temperature is typically in the range from 100 to 230° C., preferably 105 to 220° C., more preferably 110 to 210° C. and most preferably 115 to 200° C. In the case of an adiabatic reaction regime, the temperature on entry into the reactor for alternative 1 is typically in the range from 20 to 140° C., preferably 60 to 140° C., more preferably 65 to 130, most preferably 70 to 120° C., or even 75 to 110° C., and for alternative 2 in the range from 80 to 140° C., preferably 90 to 130, more preferably 95 to 120° C., most preferably 100 to 110° C., and on exit for both alternatives typically in the range from 130 to 230° C., preferably 140 to 220° C., more preferably 150 to 210° C., where the temperature on exit is always greater than on entry.

Preference is given to alternative 1 in an isothermal or adiabatic reaction regime. In the case of a corresponding adiabatic reaction regime, the heat released in the reaction of the aldehyde/ketone with the amine may already lead to a distinct rise in temperature. It is therefore possible to supply the reactor with the reactants at quite low temperatures. For example, in a continuous mode of operation, a reactant stream having a temperature of 20° C. can rise significantly as a result of the heat released in said reaction (for example to 80 or 100° C.) and hence attain the temperature required for a hydrogenation.

The process of the invention is preferably conducted continuously, with the catalyst preferably in a fixed bed arrangement in the reactor. It is possible here for the flow toward the fixed catalyst bed to be from the top or from the bottom.

The nitrogen component, based on the aldehyde group or keto group to be aminated, may be used in stoichiometric or sub- or superstoichiometric amounts.

Preferably, in the case of the amination of aldehydes or ketones with primary or secondary amines, the amine is used in a roughly stoichiometric amount or slightly superstoichiometric amount per mole of aldehyde group and/or keto group to be aminated.

The amine component (nitrogen compound) is preferably used in 0.50 to 100 times the molar amount, especially in 1.0 to 10 times the molar amount, or more preferably 1.1 to 5 times, even more preferably 1.5 to 4 times or even 2 to 3 times the molar amount, based in each case on the aldehyde groups and/or keto groups to be aminated.

Specifically ammonia is generally used with a 1.5- to 250-fold, preferably 2- to 100-fold, especially 2- to 10-fold, molar excess per mole of aldehyde group and/or keto group to be converted.

Higher excesses both of ammonia and of primary or secondary amines are possible.

In the continuous reaction regime, preference is given to running an offgas rate of 5 to 800 standard cubic meters/m³_{reactor}/h, especially 20 to 300 standard cubic meters/m³_{reactor}/h. (Standard cubic meters=volume converted to standard conditions, m³_{reactor}=reactor volume).

Hydrogen is generally used with a 1- to 50-fold, preferably 1- to 20-fold, more preferably 1.5- to 15-fold and most preferably 2- to 10-fold molar excess per mole of aldehyde group and/or keto group to be converted.

In the hydrogenation, it is also possible in principle to employ higher temperatures and higher overall pressures and catalyst hourly space velocities. The pressure in the reaction vessel which results from the sum total of the partial pressures of the nitrogen component, of the aldehyde or ketone and of the reaction products formed and of any solvent additionally used at the temperatures specified is appropriately increased to the desired reaction pressure by injecting hydrogen.

In the case of continuous operation in the liquid phase according to alternative 1, the excess aminating agent may be circulated together with the hydrogen. In an adiabatic reaction regime, accordingly, the greater the ratio of recycle stream to reactant stream, the smaller the rise in temperature.

If the catalyst is in a fixed bed arrangement, it may be advantageous for the selectivity of the reaction to mix, and to effectively “dilute”, the shaped catalyst bodies in the reactor with inert random packings. The proportion of the random packings in such catalyst preparations may be 20 to 80, particularly 30 to 60 and especially 40 to 50, parts by volume.

The water of reaction formed in the course of the reaction (in each case one mole per mole of aldehyde group or keto group converted) generally has no detrimental effect on the degree of conversion, the reaction rate, the selectivity, or the catalyst lifetime, and is therefore appropriately only removed from the resulting crude amine on workup thereof, for example by distillation.

After the reaction output has been suitably decompressed, the excess hydrogen and any excess aminating agent present are removed therefrom, and the resultant crude amine product is purified, by means of a fractional rectification for example. Suitable workup methods are described in EP 1 312 600 A and EP 1 312 599 A (both BASF AG), for example. The excess aminating agent and the hydrogen are advantageously recycled back into the reaction zone. The same applies to any incompletely converted aldehyde or ketone components.

Unconverted reactants and any suitable by-products obtained can be recycled back into the synthesis. Unconverted reactants may be passed through the catalyst bed again in batchwise or continuous mode.

### Reactants

Of the possible aldehyde and ketone reactants, preference is given to aldehydes, especially monoaldehydes (aldehydes having just one aldehyde group).

Preference is given to aliphatic (including cycloaliphatic) or aromatic aldehydes or ketones having at least 7 carbon atoms (in the case of an aldehyde) or at least 8 carbon atoms (in the case of a ketone), preferably 7 to 15 or 8 to 16 carbon atoms. Said compounds may comprise further heteroatoms, for example O, N or S, although preference is given to corresponding aliphatic or aromatic hydrocarbons that do not comprise any heteroatoms.

Preference is further given to corresponding aromatic compounds, particular preference to corresponding aromatic aldehydes.

Aminating agents in the process of the invention, as well as ammonia, are primary and secondary amines. Particular preference is given to diamines, especially primary diamines.

The process of the invention is especially suitable for preparation of an amine by reacting an aldehyde and/or ketone with a primary diamine (for example ethylene-1,2-diamine (EDA) or propylene-1,2-diamine (1,2-PDA), but also diethylenetriamine (DETA) or triethylenetetramine (TETA)).

The process of the invention is suitable, for example, for preparation of amines of the formula (A)

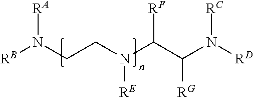

by reacting an aldehyde and/or ketone of the formula (a)

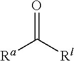

with an amine of the formula (b)

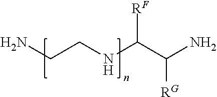

where, in the formulae (A), (a) and (b),
n is 0 to 7,
R^{a} is substituted or unsubstituted phenyl,
R^{A} is CHR^{I}R^{a},
R^{B}, R^{C}, R^{D}, R^{E} are independently R^{A} or H,
R^{F} and R^{G} (if n=1 to 7) are both H, or (if n=0) are both H or one of the two radicals is H and the other is methyl,
R^{I} is H or C₁ to C₄-alkyl.

When n=0 the amine of the formula (b) is either 1,2-EDA or 1,2-PDA, when n=1 it is DETA, for example, and when n=2 it is TETA.

Preferably, R^{a} is unsubstituted phenyl.

Preference is given to reaction with an aldehyde. In this case, R^{I} is H.

How many of the R^{B}, R^{C}, R^{D} and R^{E} radicals are H or R^{A} depends essentially on the molar ratio of amine to ketone or aldehyde. The higher the excess of amine, the more of the R^{B}, R^{C}, R^{D} and R^{E} radicals are H. The statements made above with regard to the molar amount of the amine component are correspondingly applicable.

Preferably, n is 0 to 4.

When n=0, preference is given to corresponding mixtures of amines in which R^{B} and R^{D} are H and R^{C} is either H or R^{A}. This corresponds to a mixture of amines in which just one amino group or both amino groups of the 1,2-EDA or 1,2-PDA have reacted with an aldehyde or ketone and consequently the formulae (Ia) and (Ib) shown below.

When n=1 to 7 or 1 to 4, R^{B}, R^{D} and R^{E} are preferably H. Particular preference is given to the preparation of corresponding mixtures of amines in which R^{B}, R^{D} and R^{E} are H and R^{C} is either H or R^{A}. Particular preference is given in this context to the preparation of benzyldiethylenetriamine (benzyl-DETA) and N,N′-benzyldiethylenetriamine (dibenzyl-DETA) by conversion of diethylenetriamine (DETA) and benzaldehyde, and to the preparation of benzyltriethylenetetramine (benzyl-TETA) and N,N′-benzyltriethylenetetramine (dibenzyl-TETA) by the reaction of triethylenetetramine (TETA) and benzaldehyde.

R^{I} is preferably H or methyl, more preferably (because aldehydes are preferred) H.

The process of the invention is particularly suitable for preparation of amines of the formula (Ia) or (Ib) and (Ib′)

1.

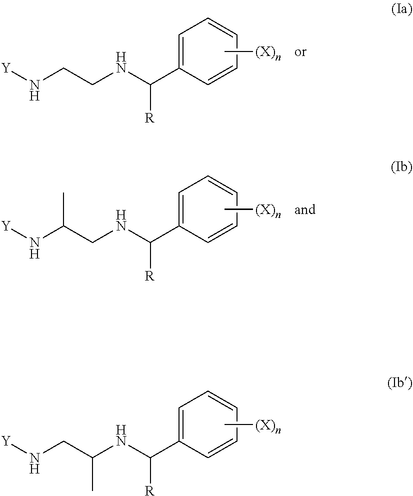

by reacting an aldehyde and/or ketone of the formula (II)

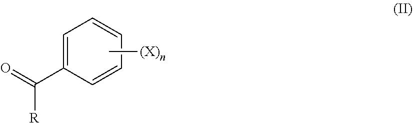

and ethylene-1,2-diamine (EDA) or propylene-1,2-diamine (1,2-PDA)
where, in the formulae (Ia), (Ib), (Ib′) and (II),
n is 0 or 1 or 2 or 3,
R is a hydrogen radical or a hydrocarbyl radical having 1 to 6 carbon atoms,
X represents identical or different radicals selected from the group consisting of alkyl,
alkoxy and dialkylamino each having 1 to 18 carbon atoms, and
Y is a hydrogen radical or a radical of the formula

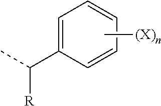

in which case the amines of the formula (Ib) and (Ib′) are identical.

The preparation of amines of the formula (Ia) is preferred.

With regard to amines of the formula (Ia), preference is given to the following features and combinations of features:

Preferably, R is a hydrogen radical or is methyl or is phenyl. More preferably, R is a hydrogen radical or is methyl, especially a hydrogen radical.

Preferably, n is 0 or 1 or 2, more preferably 0 or 1, most preferably 0.

Preferably, X represents identical or different radicals selected from the group consisting of alkyl, alkoxy and dialkylamino each having 1 to 12, especially 1 to 4, carbon atoms. More preferably, X is methyl or isopropyl or tert-butyl or methoxy or dimethylamino. Most preferably, X is methoxy or dimethylamino.

Preferably, the X radical is in meta and/or para position. In the case that n=1, the X radical is especially in para position.

Particular preference is given to an amine of the formula (Ia) in which R is a hydrogen radical and n is 0.

Particular preference is further given to an amine of the formula (Ia) in which R is a hydrogen radical, n is 1 and X is methoxy or dimethylamino in para position.

The reaction of EDA with the appropriate aldehyde or ketone of the formula (II) always gives rise both to corresponding amines of the formula (Ia) in which Y is hydrogen and those in which Y is a corresponding radical of the above formula. The ratio thereof can be adjusted via the molar ratio of EDA to aldehyde/ketone of the formula (II). The higher the excess of EDA here, the higher the proportion of amines of the formula (Ia) in which Y is hydrogen. Corresponding preferred ratios of generally amine to aldehyde/ketone are specified further up.

Particular preference is given to the preparation of amines of the formula (Ia) selected from the group consisting of N-benzylethane-1,2-diamine and N,N′-dibenzylethane-1,2-diamine, N-(4-methylbenzyl)ethane-1,2-diamine and N,N′-di(4-methylbenzyl)ethane-1,2-diamine, N-(4-isopropylbenzyl)ethane-1,2-diamine and N,N′-di(4-isopropylbenzyl)ethane-1,2-diamine, N-(4-tert-butylbenzyl)ethane-1,2-diamine and N,N′-di(4-tert-butylbenzyl)ethane-1,2-diamine, N-(4-methoxybenzyl)ethane-1,2-diamine and N,N′-di(4-methoxybenzyl)ethane-1,2-diamine, N-(4-(dimethylamino)benzyl)ethane-1,2-diamine and N,N′-di(4-(dimethylamino)benzyl)ethane-1,2-diamine, N-(1-phenylethyl)ethane-1,2-diamine and N,N′-di(1-phenylethyl)ethane-1,2-diamine, N-benzhydrylethane-1,2-diamine and N,N′-dibenzhydrylethane-1,2-diamine, N-(1-(4′-methyl)phenylethyl)ethane-1,2-diamine and N,N′-di(1-(4′-methyl)phenylethyl)ethane-1,2-diamine, and also N-(1-(4′-methoxy)phenylethyl)ethane-1,2-diamine and N,N′-di(1-(4′-methoxy)phenylethyl)ethane-1,2-diamine, from the corresponding aldehydes or ketones and 1,2-EDA. Among these, preference is given to N-benzylethane-1,2-diamine and N,N′-dibenzylethane-1,2-diamine, N-(4-methoxybenzyl)ethane-1,2-diamine and N,N′-di(4-methoxybenzyl)ethane-1,2-diamine, and also N-(4-(dimethylamino)benzyl)ethane-1,2-diamine and N,N′-di(4-(Dimethylamino)benzyl)ethane-1,2-diamine, especially N-benzylethane-1,2-diamine and N,N′-dibenzylethane-1,2-diamine.

A suitable aldehyde of the formula (II) is especially benzaldehyde, 2-methylbenzaldehyde (o-tolualdehyde), 3-methylbenzaldehyde (m-tolualdehyde), 4-methylbenzaldehyde (p-tolualdehyde), 2,5-dimethylbenzaldehyde, 4-ethylbenzaldehyde, 4-isopropylbenzaldehyde (cuminaldehyde), 4-tert-butylbenzaldehyde, 2-methoxybenzaldehyde (o-anisaldehyde), 3-methoxybenzaldehyde (m-anisaldehyde), 4-methoxybenzaldehyde (anisaldehyde), 2,3-dimethoxybenzaldehyde, 2,4-dimethoxybenzaldehyde, 2,5-dimethoxybenzaldehyde, 3,4-dimethoxybenzaldehyde (veratrum aldehyde), 3,5-dimethoxybenzaldehyde, 2,4,6-trimethylbenzaldehyde, 2,4,5-trimethoxybenzaldehyde (asarone aldehyde), 2,4,6-trimethoxybenzaldehyde, 3,4,5-trimethoxybenzaldehyde or 4-dimethylaminobenzaldehyde. Preference is given to benzaldehyde, 4-isopropylbenzaldehyde (cuminaldehyde), 4-tert-butylbenzaldehyde, 4-methoxybenzaldehyde (anisaldehyde) or 4-dimethylaminobenzaldehyde.

Suitable ketones of the formula (II) are especially acetophenone, benzophenone, 2′-methylacetophenone, 3′-methylacetophenone, 4′-methylacetophenone, 2′-methoxyacetophenone, 3′-methoxyacetophenone, 4′-methoxyacetophenone, 2′,4′-dimethylacetophenone, 2′,5′-dimethylacetophenone, 3′,4′-dimethylacetophenone, 3′,5′-dimethylacetophenone, 2′,4′-dimethoxyacetophenone, 2′,5′-dimethoxyacetophenone, 3′,4′-dimethoxyacetophenone, 3′,5′-dimethoxyacetophenone, 2′,4′,6′-trimethylacetophenone or 2′,4′,6′-trimethoxyacetophenone. Preference is given to acetophenone, benzophenone, 4′-methylacetophenone or 4′-methoxyacetophenone. Particular preference is given to acetophenone.

A particularly preferred aldehyde or ketone of the formula (II) is benzaldehyde, 4-methoxybenzaldehyde (anisaldehyde) or 4-dimethylaminobenzaldehyde. Most preferred is benzaldehyde.

In one embodiment, a mixture of two or more different aldehydes or ketones of the formula (II) is used for the reaction, especially a mixture of benzaldehyde and 4-methoxybenzaldehyde or 4-dimethylaminobenzaldehyde.

With regard to amines of the formula (Ib) and (Ib′), preference is given to the following features and combinations of features:

Preferably, n is 0 or 1 or 2, more preferably 0 or 1, most preferably 0.

Preferably, X represents identical or different radicals selected from the group consisting of alkyl, alkoxy and dialkylamino each having 1 to 12, especially 1 to 4, carbon atoms. More preferably, X is methyl or is methoxy or is dimethylamino.

Preferably, R is a hydrogen radical or is methyl, especially a hydrogen radical.

Particular preference is given to an amine of the formula (Ib) and (Ib′) in which n is 0.

Particular preference is further given to an amine of the formula (Ib) in which n is 1 and X is methoxy or is dimethylamino.

Preferably, the methoxy group or the dimethylamino group is in para position.

The reaction of 1,2-PDA with the appropriate aldehyde or ketone of the formula (II) always gives rise both to corresponding amines of the formula (Ib) and (Ib′) in which Y is hydrogen and those in which Y is a corresponding radical of the above formula. The ratio thereof can be adjusted via the molar ratio of 1,2-PDA to aldehyde/ketone of the formula (II). The higher the excess of 1,2-PDA here, the higher the proportion of amines of the formula (Ib) and (Ib′) in which Y is hydrogen. Corresponding preferred ratios of generally amine to aldehyde/ketone are specified further up.

If Y is not hydrogen, the amines of the formula (Ib) and (Ib′) are identical. With regard to those amines in which Y is hydrogen, typically more amine of the formula (Ib) than amine of the formula (Ib′) is formed. This is connected to the fact that the amino group further removed from the methyl group can react more easily with an aldehyde or ketone.

Very particular preference is given to the preparation of amines of the formula (Ib) and (Ib′) selected from the group consisting of
N¹-benzylpropane-1,2-diamine, N²-benzylpropane-1,2-diamine and N²,N²-dibenzylpropane-1,2-diamine,
N¹-(4-isopropylbenzyl)propane-1,2-diamine, N²-(4-isopropylbenzyl)propane-1,2-diamine and N¹,N²-di(4-isopropylbenzyl)propane-1,2-diamine
N¹-(4-tert-butylbenzyl)propane-1,2-diamine, N²-(4-tert-butylbenzyl)propane-1,2-diamine and N¹,N²-di(4-tert-butylbenzyl)propane-1,2-diamine
N¹-(4-methoxybenzyl)propane-1,2-diamine, N²-(4-methoxybenzyl)propane-1,2-diamine and N¹,N²-di(4-methoxybenzyl)propane-1,2-diamine
N¹-(4-(dimethylamino)benzyl)propane-1,2-diamine, N²-(4-(dimethylamino)benzyl)propane-1,2-diamine and N¹,N²-di(4-(dimethylamino)benzyl)propane-1,2-diamine
N¹-(1-phenylethyl)propane-1,2-diamine, N²-(1-phenylethyl)propane-1,2-diamine and
N¹,N²-di(1-phenylethyl)propane-1,2-diamine
N¹-benzhydrylpropane-1,2-diamine, N²-benzhydrylpropane-1,2-diamine and N¹,N²-dibenzhydrylpropane-1,2-diamine
N¹-(1-(4′-methyl)phenylethyl)propane-1,2-diamine, N²-(1-(4′-methyl)phenylethyl)propane-1,2-diamine and N¹,N²-di(1-(4′-methyl)phenylethyl)propane-1,2-diamine, and
N¹-(1-(4′-methoxy)phenylethyl)propane-1,2-diamine, N²-(1-(4′-methoxy)phenylethyl)propane-1,2-diamine and N¹,N²-di(1-(4′-methoxy)phenylethyl)propane-1,2-diamine

from the corresponding aldehydes or ketones and 1,2-PDA.

Among these, very particular preference is given to N′-benzylpropane-1,2-diamine, N²-benzylpropane-1,2-diamine and N¹,N²-dibenzylpropane-1,2-diamine.

Among these, very particular preference is also given to N′-(4-methoxybenzyl)propane-1,2-diamine, N²-(4-methoxybenzyl)propane-1,2-diamine and N¹,N¹-di(4-methoxybenzyl)propane-1,2-diamine.

Among these, very particular preference is also given to N′-(4-(dimethylamino)benzyl)propane-1,2-diamine, N²-(4-(dimethylamino)benzyl)propane-1,2-diamine and N¹,N²-(4-(dimethylamino)benzyl)propane-1,2-diamine.

In the above nomenclature, IN′ is bonded to the primary carbon atom and N² to the secondary carbon atom of the 1,2-PDA.

A suitable aldehyde of the formula (II) is especially benzaldehyde, 2-methylbenzaldehyde (o-tolualdehyde), 3-methylbenzaldehyde (m-tolualdehyde), 4-methylbenzaldehyde (p-tolualdehyde), 2,5-dimethylbenzaldehyde, 4-ethylbenzaldehyde, 4-isopropylbenzaldehyde (cuminaldehyde), 4-tert-butylbenzaldehyde, 2-methoxybenzaldehyde (o-anisaldehyde), 3-methoxybenzaldehyde (m-anisaldehyde), 4-methoxybenzaldehyde (anisaldehyde), 2,3-dimethoxybenzaldehyde, 2,4-dimethoxybenzaldehyde, 2,5-dimethoxybenzaldehyde, 3,4-dimethoxybenzaldehyde (veratrum aldehyde), 3,5-dimethoxybenzaldehyde, 2,4,6-trimethylbenzaldehyde, 2,4,5-trimethoxybenzaldehyde (asarone aldehyde), 2,4,6-trimethoxybenzaldehyde, 3,4,5-trimethoxybenzaldehyde or 4-dimethylaminobenzaldehyde.

Preference is given to benzaldehyde, 4-isopropylbenzaldehyde (cuminaldehyde), 4-tert-butylbenzaldehyde, 4-methoxybenzaldehyde (anisaldehyde) or 4-dimethylaminobenzaldehyde.

Suitable ketones of the formula (II) are especially acetophenone, benzophenone, 2′-methylacetophenone, 3′-methylacetophenone, 4′-methylacetophenone, 2′-methoxyacetophenone, 3′-methoxyacetophenone, 4′-methoxyacetophenone, 2′,4′-dimethylacetophenone, 2′,5′-dimethylacetophenone, 3′,4′-dimethylacetophenone, 3′,5′-dimethylacetophenone, 2′,4′-dimethoxyacetophenone, 2′,5′-dimethoxyacetophenone, 3′,4′-dimethoxyacetophenone, 3′,5′-dimethoxyacetophenone, 2′,4′,6′-trimethylacetophenone or 2′,4′,6′-trimethoxyacetophenone. Preference is given to acetophenone, benzophenone, 4′-methylacetophenone or 4′-methoxyacetophenone. Particular preference is given to acetophenone.

A particularly preferred aldehyde or ketone of the formula (II) is benzaldehyde, 4-methoxybenzaldehyde or 4-dimethylaminobenzaldehyde.

Most preferred is benzaldehyde.

In one embodiment, a mixture of two or more different aldehydes or ketones of the formula (II) is used for the reaction, especially a mixture of benzaldehyde and 4-methoxybenzaldehyde or 4-dimethylaminobenzaldehyde.

The examples that follow serve to elucidate the invention without restricting it in any way.

### EXAMPLES

### Example 1a: Preparation of a Catalyst Comprising Cu, Al and La

A mixture of 12.41 kg of a 19.34% copper nitrate solution, and 14.78 kg of an 8.12% aluminum nitrate solution and 1.06 kg of a 37.58% lanthanum nitrate solution×6H₂O was dissolved in 1.5 l of water (solution 1). Solution 2 includes 60 kg of a 20% anhydrous Na₂CO₃. Solution 1 and solution 2 are guided via separate conduits into a precipitation vessel that has been equipped with a stirrer and comprises 10 l of water heated to 60° C. By appropriate adjustment of the feed rates of solution 1 and solution 2, the pH was brought here to 6.2.

While keeping the pH constant at 6.2 and the temperature at 60° C., the entirety of solution 1 was reacted with soda. The suspension thus formed was then stirred for a further 1 hour, keeping the pH at 7.2 by occasional addition of dilute nitric acid or soda solution 2. The suspension is filtered and washed with distilled water until the nitrate content of the washing water was <10 ppm.

The filtercake was dried at 120° C. for 16 h and then calcined at 300° C. for 2 h. The catalyst powder thus obtained is precompacted with 18.9 g (1% by weight) of graphite. The compactate obtained is mixed with 94.6 g of Unicoat copper flakes and then mixed with 37.8 g (2% by weight) of graphite, and pressed to tablets of diameter 3 mm and height 3 mm. The tablets were finally calcined at 350° C. for 2 h.

The catalyst thus prepared has the following chemical composition:

Oxygen compounds of copper, calculated as Cu: 68% by wt.

Oxygen compounds of aluminum, calculated as Al:13% by wt.

Oxygen compounds of lanthanum, calculated as La: 11% by wt.

Elemental copper: 8% by wt.

The above concentration figures (in % by weight) are based on the total mass of the metals (Cu, Al, La).

### Example 1b: Preparation of a Catalyst Comprising Cu and Al

The catalyst was prepared by impregnating gamma-Al₂O₃ powder with an aqueous copper nitrate solution, followed by calcination. Tableting was effected by a customary method.

The catalyst thus prepared has the following chemical composition:

Oxygen compounds of copper, calculated as Cu: 64.8% by wt.

Oxygen compounds of aluminum, calculated as Al:35.2% by wt.

The above concentration figures (in % by weight) are based on the total mass of the metals (Cu, Al).

### Example 2—Continuous Preparation of N-Benzylethylene-1,2-Diamine (NBEDA)

A 6 l Miniplant reactor was used. This was charged, from the bottom upward, with 1000 ml of ceramic rings, 3500 ml of catalyst according to example 1a (referred to hereinafter as catalyst), and 1600 ml of ceramic rings. The catalyst was activated under standard pressure at a starting temperature of 180° C. with hydrogen, diluted with nitrogen. After 12 h, the temperature was increased to 200° C. Thereafter, the activation was continued with pure hydrogen at a temperature of 200° C. for a further 6 h. Subsequently, the reactor was cooled down to 70° C., hydrogen was injected up to a pressure of 100 bar, and ethylene-1,2-diamine (1,2-EDA) was fed in. Once the catalyst had been fully impregnated with 1,2-EDA, it was heated to the desired temperature. 1,2-EDA and benzaldehyde (BA) were fed into a mixing chamber in the desired ratio and guided into the reactor via a preheater. Benzaldehyde was converted completely. Further reaction parameters are shown in table 1.

Samples were analyzed by gas chromatography. This was done using an Agilent DB1 column (length: 30 m, internal diameter: 0.32 mm, layer thickness: 3.0 μm) and a flame ionization detector. The temperature program was as follows: Start at 80° C., heat to 280° C. at 10° C./min, hold at that temperature for 35 min. The respective peaks were identified by means of GC-MS (gas chromatography coupled to mass spectrometry). The respective GC area percentages were used to calculate the molar selectivity based on BA for those of the individual components.

TABLE 1

| | | | | Load | | | | | | | |
|---|---|---|---|---|---|---|---|---|---|---|---|
| | | | | based on | | Molar selectivity based on BA | | | | | |
| | Run time | T | MR | BA | MR | | | Dibenzyl— | | | Dibenzyl |
| Number | [h] | [° C.] | EDA:BA | [kg/L/h] | H₂/BA | NBEDA | DBI | EDA | BnOH | BnNH₂ | aminal |
| 1 | 86 | 120 | 2.5 | 0.20 | 9.46 | 78.0 | 0.0 | 17.0 | 4.0 | 0.5 | 0.2 |
| 2 | 147 | 139 | 1.7 | 0.29 | 6.50 | 68.7 | 0.0 | 24.7 | 5.2 | 0.2 | 0.2 |
| 3 | 1228 | 141 | 2.1 | 0.28 | 6.67 | 73.9 | 0.0 | 21.1 | 3.4 | 0.2 | 0.4 |
| 4 | 1336 | 139 | 2.2 | 0.40 | 4.73 | 74.3 | 0.0 | 20.7 | 2.7 | 0.1 | 1.1 |
| 5 | 1468 | 145 | 2.2 | 0.40 | 4.70 | 74.9 | 0.0 | 20.5 | 2.8 | 0.1 | 0.9 |
| 6 | 1708 | 145 | 2.1 | 0.15 | 12.6 | 74.6 | 0.0 | 21.3 | 2.7 | 0.3 | 0.1 |
| 7 | 1924 | 145 | 2.1 | 0.40 | 4.83 | 70.2 | 1.1 | 17.7 | 2.4 | 0.1 | 7.6 |

T: temperature
MR: molar ratio
DBI: dibenzylethylenediimine and dibenzylethylenimine
BnOH: benzyl alcohol
BnH₂: benzylamine
BA benzaldehyde
Dibenzyl—EDA: N,N′-dibenzylethylene-1,2-diamine
NBEDA N-benzylethylene-1,2-diamine

Discussion of the Results:

According to table 1, the N-benzylethylene-1,2-diamine (NBEDA) and N,N′-dibenzylethylene-1,2-diamine products of value are obtained in high yield and selectivity. At the same time, the catalyst, even after a run time of 1924 h, still has sufficient activity and hence high stability and service life. Accordingly, the catalyst is suitable even for an industrial scale process.

Comment on examples 3 to 5 that follow: An activated catalyst is understood to mean reduction thereof in a hydrogen stream at about 200° C.

### Example 3—Batchwise Preparation of NBEDA and Dibenzyl-EDA

### Example 3a

In a beaker, 20.2 g (0.34 mol) of ethylene-1,2-diamine was dissolved in 30 g of methanol, and 17.8 g (0.16 mol) of benzaldehyde was added while cooling. This mixture was divided between two electrically heated 160 ml autoclaves having a catalyst basket made of wire mesh that had a cutout for a mechanical propeller stirrer in the middle. The catalyst basket was filled with activated catalyst (5 g of 3×3 mm tablets) according to example 1b, and the autoclave was closed. After purging with nitrogen, hydrogen was injected to 20 bar. Then one autoclave was heated to 110° C. and the other to 130° C., and the hydrogen pressure at the respective final temperature was increased to 90 bar. After 12 h, the autoclaves were cooled down and decompressed. The product mixture was analyzed by GC as described in example 2.

Excess ethylenediamine was excluded from the calculation. At 110° C. the selectivity for N-benzylethylene-1,2-diamine was 47% and for N,N′-dibenzylethylenediamine was 48%; at 130° C., the selectivity for N-benzylethylene-1,2-diamine was 49% and for N,N′-dibenzylethylenediamine was 45%. The conversion of benzaldehyde was 100% in each case.

### Example 3b

By the same procedure as in example 3a, a mixture of ethylenediamine and benzaldehyde in MeOH was hydrogenated over an activated catalyst (5 g of 3×3 mm tablets) according to example 1a at 110° C. and 130° C. and analyzed. Excess ethylenediamine was excluded from the calculation. At 110° C. the selectivity for N-benzylethylene-1,2-diamine was 47% and for N,N′-dibenzylethylenediamine was 45%; at 130° C., the selectivity for N-benzylethylene-1,2-diamine was 50% and for N,N′-dibenzylethylenediamine was 44%. The conversion of benzaldehyde was 100% in each case.

### Example 4—Batchwise Preparation of N-Benzyldiethylenetriamine (Benzyl-DETA) and N,N′-Benzyldiethylenetriamine (Dibenzyl-DETA) with Two Different Molar Ratios

In a beaker, 20.0 g (0.19 mol) of diethylenetriamine (DETA) was dissolved in 15 g of MeOH, and 20.6 g (0.19 mol) of benzaldehyde was added dropwise. This mixture was introduced into an autoclave according to example 3a, and the catalyst basket was filled with 10 g of activated catalyst according to example 1a. Hydrogenation was effected as described at 90 bar and 130° C. for 12 h. The crude mixture was analyzed by gas chromatography.

This was done using an Agilent RTX-5 Amine column (length: 30 m, internal diameter: 0.32 mm, layer thickness: 1.5 μm) and a flame ionization detector. The temperature program was as follows: Start at 60° C., heat to 280° C. at 6° C./min, hold at that temperature for 28 min. The respective peaks were identified by means of GC-MS (gas chromatography coupled to mass spectrometry). The respective GC area percentages were used to calculate the molar selectivity based on DETA for those of the individual components.

The conversion was 83%; the selectivity for benzyl-DETA based on DETA was 72% and for dibenzyl-DETA 23%.

In a beaker, 15 g (0.15 mol) of diethylenetriamine (DETA) was dissolved in 15 g of MeOH, and 30.9 g (0.29 mol) of benzaldehyde was added dropwise. This mixture was introduced into an autoclave according to example 3a, and the catalyst basket was filled with 10 g of activated catalyst according to example 1a. Hydrogenation was effected as described at 90 bar and 130° C. for 12 h. The crude mixture was analyzed by gas chromatography as described above. The conversion was 99%; the selectivity for benzyl-DETA based on DETA was 41% and for dibenzyl-DETA 49%.

### Example 5—Batchwise Preparation of N-Benzyltriethylenetetramine (Benzyl-TETA) and N,N′-Benzyltriethylenetetramine (Dibenzyl-TETA) with Two Different Molar Ratios

In a beaker, 23 g (0.16 mol) of triethylenetetramine (TETA) was dissolved in 15 g of MeOH, and 17 g (0.16 mol) of benzaldehyde was added dropwise. This mixture was introduced into an autoclave according to example 3a, and the catalyst basket was filled with 10 g of activated catalyst according to example 1a. Hydrogenation was effected as described at 90 bar and 130° C. for 12 h. The crude mixture was analyzed.

This was done using an Agilent RTX-5 Amine column (length: 30 m, internal diameter: 0.32 mm, layer thickness: 1.5 μm) and a flame ionization detector. The temperature program was as follows: Start at 120° C., heat to 280° C. at 8° C./min, hold at that temperature for 50 min. The respective peaks were identified by means of GC-MS (gas chromatography coupled to mass spectrometry). The respective GC area percentages were used to calculate the molar selectivity based on TETA for those of the individual components.

The conversion was 77%; the selectivity for benzyl-TETA based on TETA was 68% and for dibenzyl-TETA 18%.

In a beaker, 21 g (0.14 mol) of triethylenetetramine (TETA) was dissolved in 15 g of MeOH, and 23 g (0.22 mol) of benzaldehyde was added dropwise. This mixture was introduced into an autoclave according to example 3a, and the catalyst basket was filled with 10 g of activated catalyst according to example 1a. Hydrogenation was effected as described at 90 bar and 130° C. for 12 h. The crude mixture was analyzed by gas chromatography as above. The conversion was 91%; the selectivity for benzyl-TETA based on TETA was 59% and for dibenzyl-TETA 27%.

### Example 6—Continuous Preparation of N-Benzylethylene-1,2-Diamine and N,N′-Dibenzylethylenediamine in the Gas Phase (Comparative Experiment)

It should be noted that a batchwise reaction in the gas phase is not implementable for technical reasons. Accordingly, the reaction here is continuous.

A vertical oil-heated jacketed glass reactor of length 1 m and having diameter 40 mm was charged with 200 ml of steel mesh rings having diameter 5 mm, then 100 ml of a catalyst according to example 1b (3×3 mm tablets) and a further 700 ml of mesh rings. The catalyst was reduced in a hydrogen stream at up to 230° C. for 12 h.

At the lower end of the reactor was mounted a flask with a reflux condenser on top, which was provided with a tap to discharge liquid reaction product. The reactor was equipped with a pump for liquid reactant and a conduit for the blowing-in of heated hydrogen. The feeds were guided to the reactor inlet at the upper end and brought to the desired temperature on the first bed of mesh rings and mixed thoroughly.

The reactor was heated to 180° C. and charged with 593 l (STP)/h of hydrogen. Then a mixture of 29.7% ethylenediamine and 26.1% benzaldehyde in MeOH, corresponding to the composition of the mixture that had been hydrogenated batchwise in example 3a, was pumped in at a metering rate of 19 g/h every hour, which corresponded to a space velocity of 0.05 kg/I/h benzaldehyde. Samples were taken every 1 h. After the sampling after 2 h, the reactor temperature was lowered to 175° C.

The sample after 2 h was analyzed as described in example 2. Excess ethylenediamine was excluded from the calculation. The conversion of benzaldehyde was 100%. The molar selectivity for N-benzylethylene-1,2-diamine was about 2%; the selectivity for N,N′-dibenzylethylene-1,2-diamine was about 0%. Numerous by-products, some of them unidentified, were formed, and so it is not possible to state the molar selectivity with the same accuracy as above.

After the temperature had been lowered to 175° C., a sample was taken after 2 h, which had a selectivity for N-benzylethylene-1,2-diamine of about 13% and a selectivity for N,N′-dibenzylethylene-1,2-diamine of about 0.2%. The conversion of benzaldehyde was 100%. Then the temperature was lowered to 170° C. However, condensation of intermediates and products occurred here in the catalyst bed, and the experiment was stopped.

It is apparent that the preparation in the gas phase does not give satisfactory results. By comparison, the results according to example 3a show that a reaction in the liquid phase gives very good selectivities for the N-benzylethylene-1,2-diamine and N,N′-dibenzylethylene-1,2-diamine products of value.

1.-15. (canceled)

16. A process for preparing an amine by reacting an aldehyde and/or ketone with a nitrogen compound selected from the group consisting of ammonia and primary and secondary amines, and subsequent hydrogenation of the resulting reaction product in the liquid phase and in the presence of hydrogen and a heterogeneous copper oxide hydrogenation catalyst at a temperature of 20 to 230° C., wherein the aldehyde and/or ketone is reacted with the nitrogen compound either together with the hydrogenation in the liquid phase and in the presence of the hydrogen and of the catalyst (alternative 1) or in a step preceding the hydrogenation (alternative 2), and wherein the catalytically active composition of the catalyst, prior to reduction thereof with hydrogen, comprises at least 24% by weight of oxygen compounds of copper, calculated as Cu.

17. The process according to claim 16, wherein the catalytically active composition of the catalyst, prior to reduction thereof with hydrogen, comprises in the range from 24% to 98% by weight of oxygen compounds of copper, calculated as Cu.

18. The process according to claim 17, wherein the catalytically active composition of the catalyst, prior to reduction thereof with hydrogen, comprises in the range from 0.5% to 75% by weight of oxygen compounds of aluminum, calculated as Al.

19. The process according to claim 17, wherein the catalytically active composition of the catalyst, prior to reduction thereof with hydrogen, comprises in the range from 0.5% to 40% by weight of at least one oxygen compound selected from the group consisting of oxygen compounds of lanthanum, tungsten, molybdenum, titanium and zirconium, calculated as La, W, Mo, Ti and Zr.

20. The process according to claim 16, wherein the catalytically active composition of the catalyst comprises in the range from 0.1% to 40% by weight of elemental copper and/or in the range from 0.1% to 40% by weight of at least one oxygen compound selected from the group consisting of oxygen compounds of magnesium, calcium, silicon and iron, calculated as Mg, Ca, Si and Fe.

21. The process according to claim 17, wherein the copper oxide catalyst, prior to reduction thereof with hydrogen, is preparable by a process in which
i) an oxidic material comprising oxygen compounds of copper and of aluminum and at least one oxygen compound selected from the group consisting of oxygen compounds of lanthanum, tungsten, molybdenum, titanium and zirconium, is provided,
ii) pulverulent metallic copper, copper flakes, pulverulent cement or a mixture thereof, preferably pulverulent metallic copper, copper flakes or a mixture thereof, is added to the oxidic material,
iii) the mixture resulting from (ii) is shaped to give the copper oxide catalyst and is preferably then calcined at least once.

22. The process according to claim 21, wherein graphite is added to the oxidic material and/or the mixture resulting from (ii) in a total amount of 0.5% to 5% by weight, based on the total weight of the oxidic material.

23. The process according to claim 16, wherein the amine is prepared by reacting the aldehyde and/or ketone and the nitrogen compound together with the hydrogenation in the liquid phase and in the presence of hydrogen and of the catalyst.

24. The process according to claim 16, which is conducted continuously, and the catalyst hourly space velocity is in the range from 0.05 to 5 kg of aldehyde and/or ketone (alternative 1) or reaction product (alternative 2) per liter of catalyst (bed volume) and hour.

25. The process according to claim 16, wherein the nitrogen compound is used in 0.9 to 100 times the molar amount based on the aldehyde groups and/or keto groups to be aminated.

26. The process according to claim 16, wherein the hydrogenation is conducted at an absolute pressure in the range from 1 to 30 MPa.

27. The process according to claim 16, which is conducted continuously and the hydrogenation is effected in a reactor either isothermally or adiabatically, wherein, in the case of an isothermal reaction regime, for both alternatives, the temperature is in the range from 100 to 230° C., and, in the case of an adiabatic reaction regime, the temperature on entry into the reactor for alternative 1 is in the range from 20 to 140° C. and for alternative 2 is in the range from 80 to 140° C., and on exit for both alternatives is in the range from 130 to 230° C., with the temperature on exit always being greater than on entry.

28. The process according to claim 16, for preparation of amines of the formula (A) 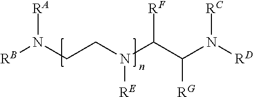
by reacting an aldehyde and/or ketone of the formula (a) 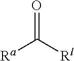
with an amine of the formula (b) 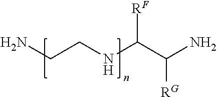
where, in the formulae (A), (a) and (b),
n is 0 to 7,
R^{a} is substituted or unsubstituted phenyl,
R^{A} is CHR^{I}R^{a},
R^{B}, R^{C}, R^{D}, R^{E} are independently R^{A} or H,
R^{F} and R^{G} (if n=1 to 7) are both H, or (if n=0) are both H or one of the two radicals is H and the other is methyl,
R¹ is H or C₁ to C₄-alkyl.

29. The process according to claim 28, for preparation of amines of the formula (Ia) or (Ib) and (Ib′) 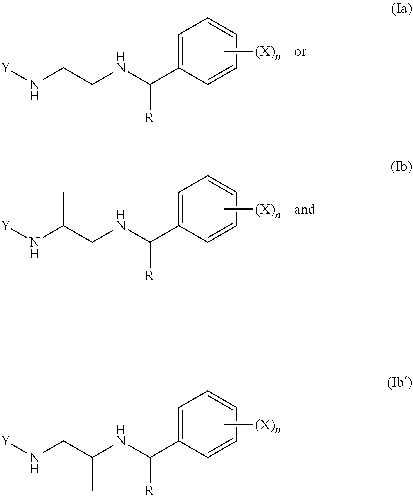
by reacting an aldehyde and/or ketone of the formula (II) 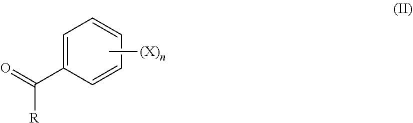
and ethylene-1,2-diamine (EDA) or propylene-1,2-diamine (1,2-PDA) where, in the formulae (Ia), (Ib), (Ib′) and (II),
n is 0 or 1 or 2 or 3,
R is a hydrogen radical or a hydrocarbyl radical having 1 to 6 carbon atoms,
X represents identical or different radicals selected from the group consisting of alkyl, alkoxy and dialkylamino each having 1 to 18 carbon atoms, and
Y is a hydrogen radical or a radical of the formula 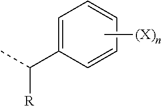
in which case the amines of the formula (Ib) and (Ib′) are identical.

30. The process according to claim 29, for preparation of N-benzylethylene-1,2-diamine (NBEDA) and N,N′-dibenzylethylene-1,2-diamine or N-benzylpropylene-1,2-diamine (NBPDA), N′-benzylpropylene-1,2-diamine and N,N′-dibenzylpropylene-1,2-diamine by reaction of benzaldehyde and EDA or 1,2-PDA.
